(19) United States
(12) Patent Application Publication
(10) Pub. No.: US 2010/0158842 A1
(43) Pub. Date: Jun. 24, 2010

(54) NATURAL SKIN WHITENER: 4-HYDROXY-OXINDOLE-3-ACETIC ACID

(75) Inventor: John Jacob WILLE, Jr., Chesterfield, NJ (US)

Correspondence Address: William H. Dippert;Eckert Seamans Cherin & Mellott, LLC, U.S. Steel Tower, 600 Grant Street, 44th Floor, Pittsburgh, PA, 15219, US

(73) Assignee: SPECTRUM DYNAMICS LLC

(21) Appl. No.: 12/576,570

(22) Filed: Oct. 9, 2009

Related U.S. Application Data
(60) Provisional application No. 61/195,697, filed on Oct. 9, 2008.

(51) Int. Cl. A61K 8/49; C07D 209/34; A61K 31/405; A61P 17/00; A61Q 19/02; A61Q 15/00

(52) U.S. Cl. 424/65; 548/486; 514/418

(57) ABSTRACT

A cosmetic and dermatological composition comprises 2, 3-dihydro-4-hydroxy-2-oxo-1-H-indole-3-acetic acid (4-hydroxy-1-oxindole-3-acetic acid) as an active ingredient for use as a melanin inhibitor and as an anti-tyrosinase enzyme inhibitor. This composition is excellent as a skin whitening agent. Since it also has potent antioxidant and anti-irritant activities it is useful in a cosmetic composition for improving hyperpigmentation disorders of the skin.

### CROSS REFERENCE TO RELATED APPLICATIONS

This application claims priority from Provisional Application Ser. No. 61/195,697 filed Oct. 9, 2008, entitled “A new Natural Skin whitener: 4-Hydroxy-Oxindole-3-Acetic Acid”.

### BACKGROUND OF THE INVENTION

The attempt to solve the problem of enzymatic browning of natural food products has led to many approaches of potential use in personal care and cosmetic applications where there is an unmet need for natural, safe and effective skin de-pigmentation agents (FAO, 2000). Among the inhibitors commonly employed are reducing and sulfating agents including L-ascorbic acid (vitamin C), erythorbic acid, cysteine, and glutathione, and acidulants such as citric acid, chelators such EDTA, phosphates, maltol, kojic acid, complex agents such as chitosan, cyclodextrans, enzyme inhibitors such as 4-hexylresorcinol, halide salts, amino acid, peptides and proteins, aromatic carboxylic acids, and aliphatic their alcohols. Indeed, a number of these have found their way in to current skin whitening products. A review of current skin whitening agents can be accessed at website: http://en.wikipedia.org/wiki/Skin_whitening.

Historically, the antioxidant quinones, such as azelaic acid and particularly, hydroquinone has been the active ingredient for inhibiting melanin production in most topical skin whitening products. Unfortunately, because of its skin irritation at useage levels higher than 4 per cent, and its potential human toxicity and fears of cancer risk, it has been banned in some EU countries. In addition, hydroquinone is an unstable ingredient in many cosmetic formulation when exposed to sun or air turning a brownish color. Furthermore, there are new derivatives of hydroquinone derived from bearberry extracts. The most potent of these skin lighteners are the arbutins (alpha-arbutin, beta-arbutin, and dexyarbutin); technically these are called hydroquinone-beta-D-glucoside. There are numerous botanical sources of arbutin including cranberry, mulberry, or blueberry shrub leaves. Neis et al. reports in U.S. Pat. No. 7,431,949 to have combined alpha-arbutin and bearberry extract with octyl stearate and polyolprepolymer-2 in the form of liposomes, claiming better skin lightening due to deeper skin penetration.

There is a growing list other botanical extracts with potential skin whitening activity including oral supplements from pomegranate and licorice, and purified compounds such as ellagic acid, ferrulic acid, and vitamin E, which all inhibit melanin production. Also, the alpha-hydroxy acids, lactic acid and glycolic acid at concentrations from 4 to 15% are thought to inhibit melanin production. The pyrole compound kojic acid, a by-product of rice fermentation, has been found to be an effective inhibitor of melanin production. Like hydroxyquinone, kojic acid is an unstable ingredient in cosmetic formulations that discolors upon exposure to air and sunlight. Nagai et al., disclose in U.S. Pat. No. 4,369,174 that a kojic acid ester with an aliphatic carboxylic acid improves skin whitening in a cosmetic composition. Others attempts to find new sources of topical skin whiteners include botanical extracts from acerola cherry fermentates (U.S. Pat. No. 5,747,006). Focus has been on finding tyrosinase inhibitors as in U.S. Pat. No. 7,125,572 derived from lemon peel extracts. Nagamine et al., in U.S. Pat. No. 7,192,617, disclose a skin whitening agent in Camu Camu seeds, and Shephard in U.S. Pat. No. 7,226,583 report compositions containing leukocyte extracts in combination with kojic acid and a multitude of supposed skin whitening agents in various botanical extracts. Skin whitening activities have been disclosed in extracts from asparagus (U.S. Pat. No. 7,247,321), black yeast, which is claimed to possess a melanin degrading enzyme (U.S. Pat. No. 7,291,340) and extracts of the roots of black cohosh are claimed to suppress melanogenesis (U.S. Pat. No. 7,364,759). Nawamura et al., in U.S. Pat. No. 7,510,716, disclose extracts from several different basidiomycetes which are strong antioxidants and have tyrosinase inhibitor activity. A topical depigmenting composition comprising a new processing method for producing extracts from Bellis perennis L has been disclosed in U.S. Pat. No. 7,527,811, and a topical skin lightening composition derived from extracts of Perilla leaves is disclosed in U.S. Pat. No. 7,544,351. A composition that contain 5,15-diacetyl-3-benzoyllathyro derived from extracts of Euphorbia Lathyris is claimed as a melanin production inhibitor in U.S. Pat. No. 6,838,481. Other plant extracts that inhibit melanin synthesis contain isoflavones such as 8-hydroxydaidzin. A topical composition for treating hyperpigmentation disorders comprising a mixture of phytic acid, a heavy metal ion chelator, and vismia with antioxidant activity is claimed in U.S. Pat. No. 7,494,643. Yamaguchi et al. U.S. Pat. No. 7,355,063 discloses a diterpene compound derived by biotransformation of Shogaol that inhibits tyrosinase activity. Finally, Shonrock et al., in U.S. Pat. No. 7,498,310 disclose cosmetic and dermatological preparations comprising oligopeptides for lightening skin where the structure of the peptide is based on Val-Val-Arg-Prol SEQ ID NO:1 as a homo-or heterodimer.

One aim of the present invention is to provide a new all natural skin whitening agent that is safe and highly effective. The search for new natural skin whiteners with improved safety and efficacy commands a high priority in world-wide personal and skin care markets. To met this challenge and satisfy the need for green/ecocitizenship, cosmetic companies have scoured the planet for more exotic plants and places. Our search began nearer to home with ubiquitous commercial food plants as corn and other grains. The effort began with the inventor's discovery that corn tassels produce a fragrant odor during pollination. Over the past decade, corn tassel extracts (Tasselin) were prepared and analyzed for their primary constituents. Biochemical and biological assessments have demonstrated that corn tassel extracts have potent anti-irritant, and anti-oxidant activities (Wille, J J. J. Invest. Dermatol. 129(4): S.I.D, Abstract, 2009). The methods for producing anti-irritant corn tassel extracts and skin care formulations for treating and or alleviating skin irritation are disclosed in U.S. Pat. No. 7,399,493. The disclosure in this patent, and the contents of the references cited therein, are hereby incorporated by reference in this specification.

### SUMMARY OF THE INVENTION

The basis of the present invention is the discovery of biological activity present in corn tassels extracts, namely, an inhibitor of melanogensis, and presumptive skin whitening agent. One of the objects of the present invention therefore, is to determine the chemical identity of the skin whitening agent present in crude corn tassel extracts. This was accomplished through thorough high pressure liquid chromatography (HPLC) analyses of hydroalcoholic fractions of corn tassels, which revealed many phenolic compounds. Further HPLC analysis was carried out to determine the chemical identity of the predominant phenolic component, which we identified as 4-hydroxy-oxindole-3-acetic acid, herein designated Tasselin A. A preliminary search on the U.S. PTO patent website failed to find any reference to 4-hydroxy-oxindole-3-acetic acid. Still, yet another aim was to determine the exact molecular weight and confirm the structure of the principal phenolic component as being 4-hydroxy-oxindole-3-acetic acid. This was verified through the technique of mass spectroscopic analysis. For the purpose of providing information necessary for cosmetic and dermatological formulation, where larger amount of the compound are required, methods were developed for isolation and purification and for assessment of it's biochemical properties and biological activities.

### The Composition

According to the invention, therefore, a composition of matter comprises a novel natural botanical compound, 4-hydroxy-oxindole-3-acetic acid, which is derived from corn tassel extracts. This compound has been shown to have multifunctional skin benefits including potent anti-irritant, anti-oxidant and excellent tyrosinase inhibitor activity suitable for use in skin whitening preparations, and in formulations for this purpose provided herein.

### The Method of Making the Composition

There is also disclosed here a method of making a cosmetic ingredient with the chemical composition and structure of 4-hydroxy-oxindole-3-acetic acid. The chemical compound was identified in extracts of corn tassels. In particular, seasonally aromatic corn tassels were harvested as previously described in U.S. Pat. No. 7,399,493 with particular attention to harvesting the tassel at the peak time of aroma production. They were collected simply by shaking the tassels directly into collecting sacks conveyed to the laboratory where they are immersed directly into ice-cold absolute alcohol and extracted for 24-48 hours at 0-4° C. Crude alcohol extracts have a light green color and a fragrance reminiscent of bee's wax. The extract is further clarified low speed centrifugation followed by filtration through a 0.4 micron nitrocellulose filter and the resulting sterile solution stored at −20° C. until further needed or then concentrated by vacuum rotary evaporation to obtain a slightly viscous mass. This material served as the starting point for chemical analysis. Dilutions of the ice-cold crude extract was submitted to preliminary assessment of its anti-oxidant activity. High pressure liquid chromatography (HPLC) was employed to determine the principle phenolic components of the tassel extract. The main component was purified by preparative HPLC, the compound as having a molecular weight of 207 daltons and the chemical structure as 4-hydroxy-oxindole-3-acetic acid. For simplicity in terminology we name this chemical compound Tasselin A. As expected from its extraction profile, Tasselin A is both water and methanol/ethanol soluble. Tasselin A is similar to several oxidation products of auxin (indole-3-acetic acid, IAA), a ubiquitous plant hormone, previously described in the scientific literature. It is thought to be an inactive metabolic waste product of auxin metabolism in Zea mays seedlings, tissues and in germinating seeds of Scots pines (Nonhebel and Bandurski, 1984; Nonhebel, 1986; Ernsted et al, 1987; Reinecke and Bandurski, 1988). Oxindole-3-acetic acid the 3-hydroxyl derivatives have been isolated from a byproduct of corn starch manufacturing plant and converted to the 3-hydroxy derivative in the presence of metal ion (Niwa et a, 2003). The 4-hydroxy derivative of oxindole is not common and has not been associated with male reproductive plant tissues in the literature before.

Purified Tasselin A has been assayed and determined, by a free-radical scavenging assay, that it has good antioxidant activity. Tests have shown that it possesses anti-irritant activity of purified Tasselin A by determining its ability to inhibit skin irritation induced by 24-hour occlusion of human forearm skin. Early in the investigation, the observation was made that partially purified corn tassel extracts inhibit melanin formation in common bread mold cultures. Studies have now been completed, demonstrating that a purified indole compound (Tasselin A) is an effective inhibitor of mushroom tyrosinase enzyme, a key enzyme in the melanin biochemical pathway.

A principal object of the present invention is therefore to provide for cosmetic and dermatological formulations containing effective dosages of Tasselin A. To this end, several cosmetic and dermatological compositions are provided, all of which are capable of topical delivery of the Tasselin A for a variety of skin lightening treatments and skin de-pigmentation disorders.

### BRIEF DESCRIPTION OF THE DRAWINGS

FIG. 1. High pressure liquid chromatography (HPLC) profile of the bulk phenolic compounds in alcoholic extracts of corn tassel pollen sacs.

FIG. 2. Preparative HPLC profile of principal component (fraction 3) of the phenolic compounds found in alcohol extracts of corn tassels pollen sacs.

FIG. 3. LC-MS data on chemical composition of Tasselin A.

FIG. 4. Free-radical scavenging activity of purified Tasselin A in the diphenylhydrazine (DPPN) assay.

FIG. 5. Purified Tasselin A suppresses skin irritation.

FIG. 6. Corn tassel extracts inhibit mold melanization.

FIG. 7. Purified Tasselin A inhibits tyrosinase enzyme activity.

### DETAILED DESCRIPTION OF THE PREFERRED EMBODIMENTS

### Example 1

Isolation and extraction of Tasselin A.

Tassels are the male reproductive organs of the corn plant. Pollen develops and ripens within the tassel in temporal synchrony with the development of ripening female ovarian germ cells at approximately 6-8 weeks after planting. At this stage of tassel development there is a distinctive change in color of the tassels from green to reddish-brown and development of a unique and distinctive aroma. This distinctive aroma persists only during the stage of pollen release (Wille, U.S. Pat. No. 7,399,439).

Tassels collected in the field at this stage of pollen maturation and release are enriched in scented pollen sacs. These pollen sacs are the starting material for isolation of Tasselin A compound. They are collected simply by shaking the tassels directly into collecting sacks conveyed to the laboratory where they are immersed directly into ice-cold absolute alcohol and extracted for 24-48 hours at 0-4° C. The ice-cold extract is clarified by low speed centrifugation and stored at −20° C. until needed further purification steps.

Corn tassels collected from field corn either lack or have reduced seasonal aroma and correspondingly have negligible extractable amounts of Tasselin A. Many different batches of corn tassels were collected from widely separate regions of North America during their appropriate growing season and corn plant maturation. Corn plants derived from varieties of sweet corn seeds were found to not only produce seasonally aromatic corn tassel but also yield Tasselin A as the main phenolic compound upon alcohol extraction. This of critical importance for any future commercialization of Tasselin A.

### Example 2

### Chemical Identification of Tasselin A

Over the course of several years many different Tasselin A extracts were prepared and examined in collaboration with scientists at the United States Department of Agriculture in Peoria, Ill. A brief abstract of this work was published ([CITE], Wille and Berhow, J. Invest. Dermatol. April, 2009).

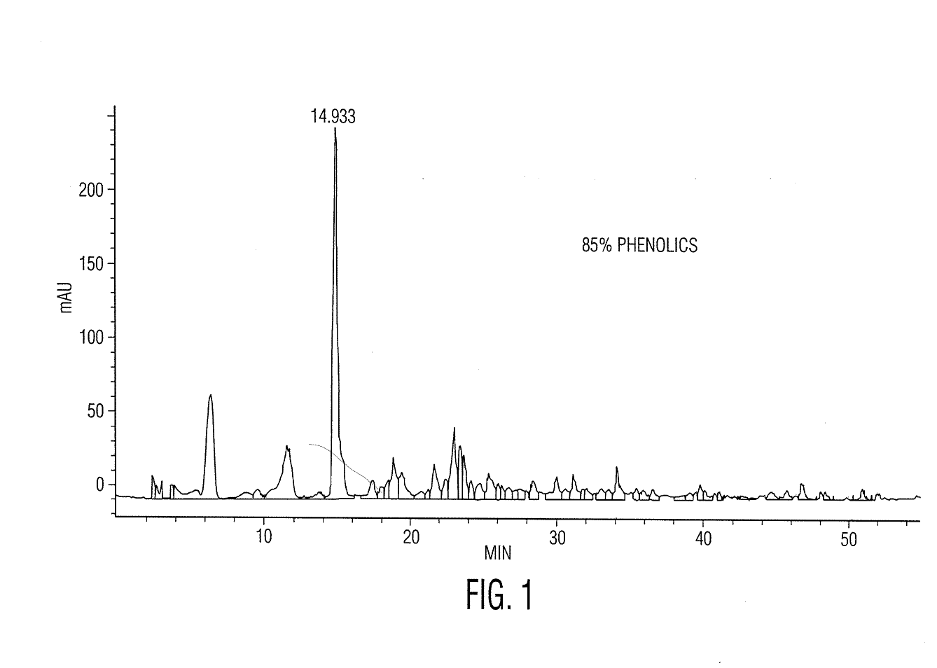

The alcoholic extracts were investigated by the technique of high pressure liquid chromatography (HPLC). A typical chromatographic profile is shown in FIG. 1.

Area under the curve (AUC) measurements have shown that phenolic compounds comprise greater than 80 per cent of the physical mass chromatographed. Many of which were matched to known phenolic compounds. However, the main component, which consistently eluted at approximately 15 minutes, and which is of interest to this invention was an as yet unidentified compound.

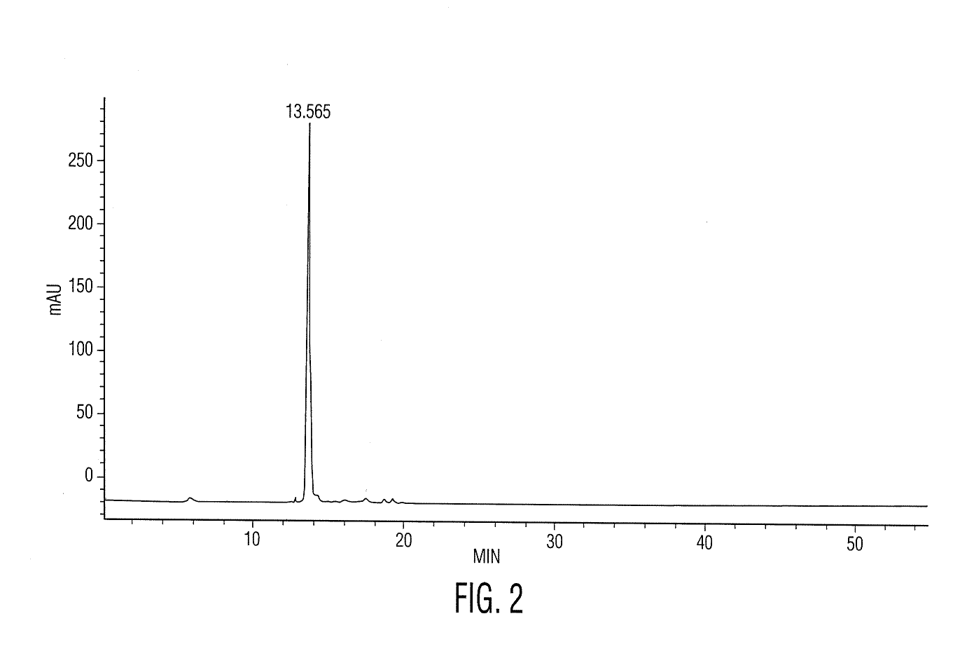

Positive identification proceeded by first isolating the main phenolic component by taking a “cut” or fraction encompassing the main peak of absorbance by the method of preparative HPLC. This material was pooled and rerun by the method of preparative HPLC. FIG. 2 presents the HPLC profile of the isolated peak material. It shows a hyperfine peak that is representative of a highly purified compound.

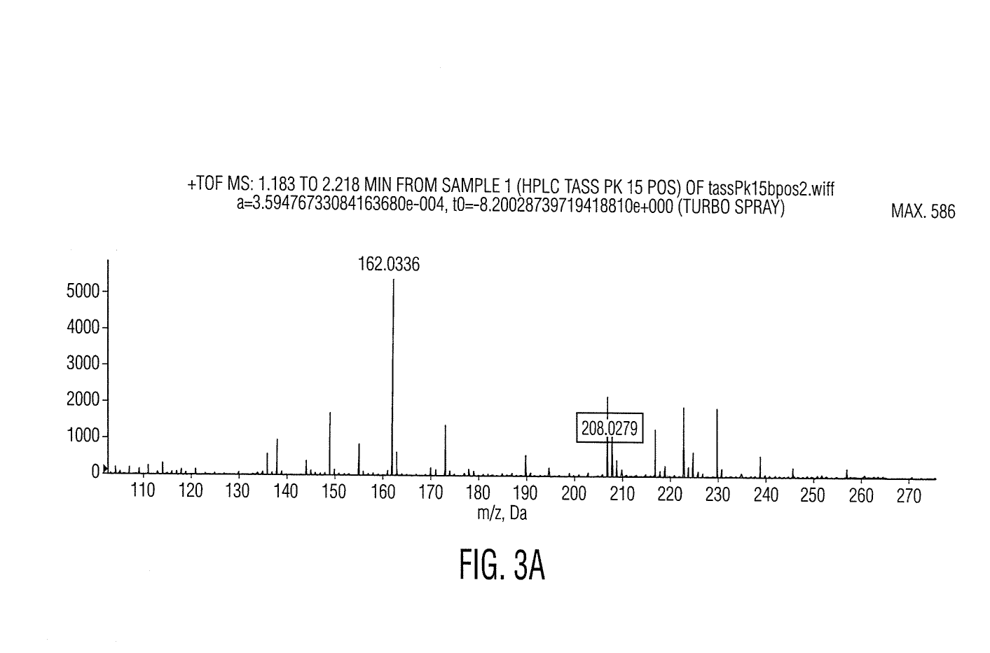

The main component purified by preparative HPLC (FIG. 2), was further analysed by LS-MS mass spectroscopy to determine the molecular structure (FIG. 3). Previous work on milligram quantities of preparative HPLC purified Tasselin A had identified a compound with a putuative molecular weight of 207 daltons. Of the many possible candidates with an approximate MW of 207, is an indole type compound based on a published report that crude Tasselin extracts had anti-irritant activity similar to the plant hormone, indole-3-acetic acid ([CITE] Wille J, 2003; Wille J: U.S. Pat. No. 7,399,439).

In order to determine the exact molecular weight and molecular structure, detailed mass spectrometry was conducted. FIG. 3 and Table I shows the results of chemical analysis so performed. It identified Tasselin A with a molecular weight of 207 daltons, and a molecular formula of C10H9NO4. Table II shows the molecular structure of Tasselin A and identifies it as 2,3-dihydro-4-hydroxy-2-oxo-1H-indole-3-acetic acid with a synonym name as 4-hydroxy-oxindole-3-acetic acid. Table II also shows the molecular formula and the percentage composition. For simplicity in terminology we designate this chemical compound as Tasselin A.

### Example 3

### Antioxidant Activity of Tasselin A

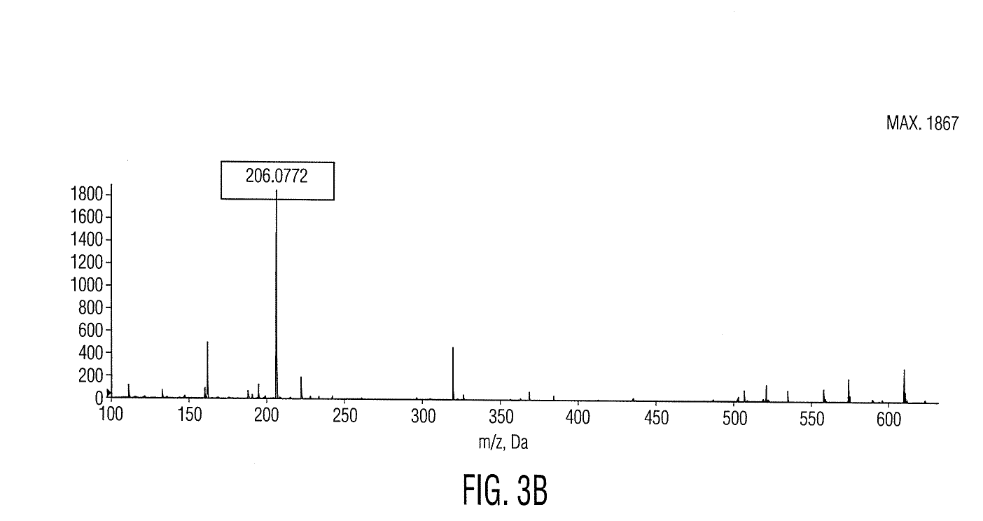

Purified Tasselin A has been assayed and determined its free-radical scavenging activity. FIG. 4 present typical results of standardized 1,1-diphenyl-2-picrylhydrazyl radical (DPPH*) reagent test as previously described (Bonina et al, 2002). This test was previously disclosed and employed on crude corn tassel alcoholic extracts (Wille J J: U.S. Pat. No. 7,399,439). Here we confirm that purified Tasselin A has potent antioxidant activity. A 1.0 mg/ml stock solution of purified Tasselin A in ethanol was diluted with anhydrous ethanol to a final concentration of 100 microgram per milliliter and assayed in the DPPH* free-radical scavenging assay. As shown in FIG. 4, there was a 40% reduction in absorbance measured at 595 nm, indicating that Tasselin A has good anti-oxidant activity.

### Example 4

### Anti-Irritant Activity of Tasselin A

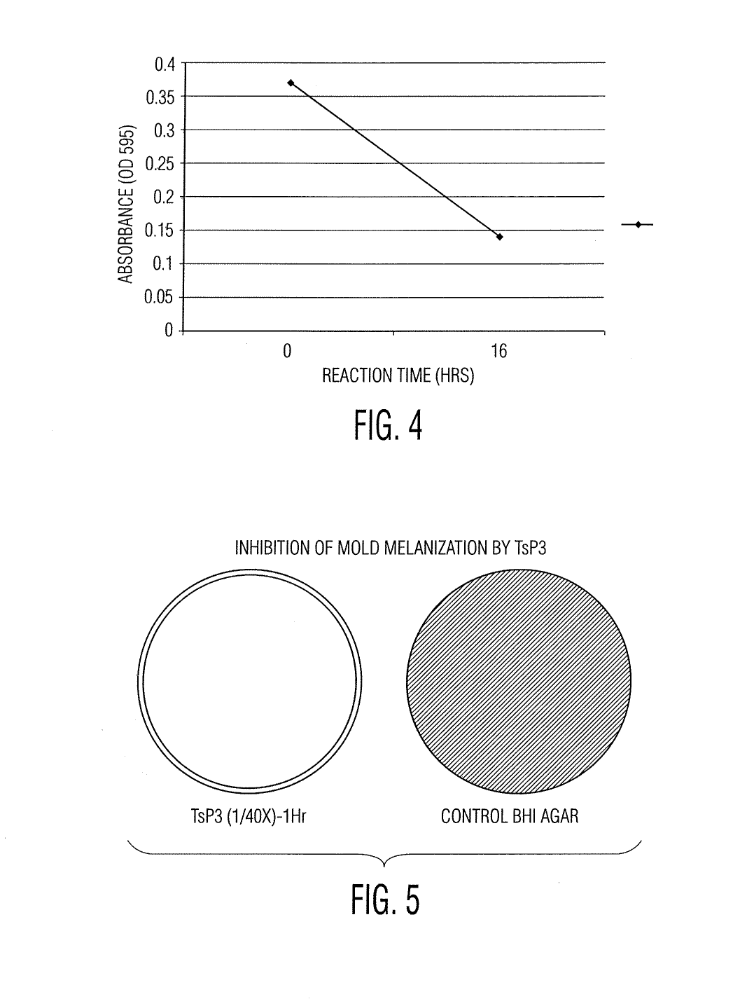

The anti-irritant activity of purified Tasselin A has been assessed by determining its ability to inhibit skin irritation induced by 24-hour occlusion of human forearm skin with a solution of 0.4% benzalkonium chloride (BC) with or without inclusion of 0.01% (100 micrograms/milliliter) of purified Tasselin A. FIG. 5 is a photograph of the volar surface of a subject forearm immediately after removal of the occlusive dressing. There are two circled skin areas: A) control site, and B) test site. Visual scoring of the control area show that there was an irritant reaction accompanied by swelling and erythema (redness). By contrast, the test site was no different than untreated adjacent skin, i.e., no swelling or redness. This demonstrates that Tasselin A can prevent skin irritation when co-administered with an irritating concentration of a known irritant (0.5% benzalkonium chloride).

### Example 5

### Inhibition of Melanin Production in Mold Cultures by Corn Tasselin Extracts:

A stock culture of bread mold was prepared by growth spores in a sterile phosphate buffered saline solution containing corn starch (4%, w/v) for 10 days at 37° C. The control portion of the stock culture was diluted one part to four with pre-warned PBS. The treatment portion was also split one part to four with pre-warmed PBS and a 10% (v/v) dilution of a corn tassel extract in 70% (v/v) ethanol solution added. The control and treatment working stock solutions were incubated for one hours at 37° C., and then plated on Beef Heart Infusion nutrient agar. The cultures were periodically examined over a two week period. During the first week both cultures grew extensively, but by the second week of inspection, it was noticed that the control culture formed melanized spores while no melanization occurred in the treated culture.

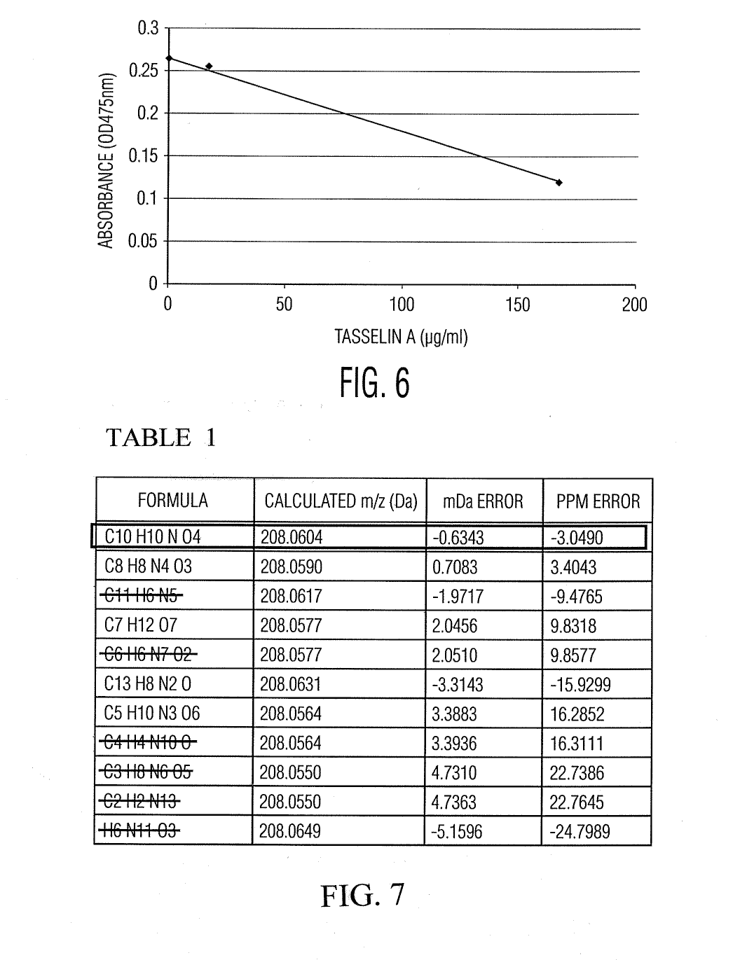

FIG. 6 is a photograph of the control and treated plates. The result suggests that crude corn tassel extracts do contain an inhibitor of melanin production. In fact, this idea was further strengthened when the molecular structure of purified Tasselin A became known. Tis is shown by reference to it's similarity to the chemical structure of the pentultimate precursor metabolic intermediates of melanin synthesis in the tryptophan pathway, e.g., indole-5,3-quinone. It is likely then that 4-hydroxy-1-oxindole is a competitive inhibitor of the key enzyme reactions in the melanin synthesis pathway.

### Example 6

### Inhibition of Tyrosinase by Tasselin A

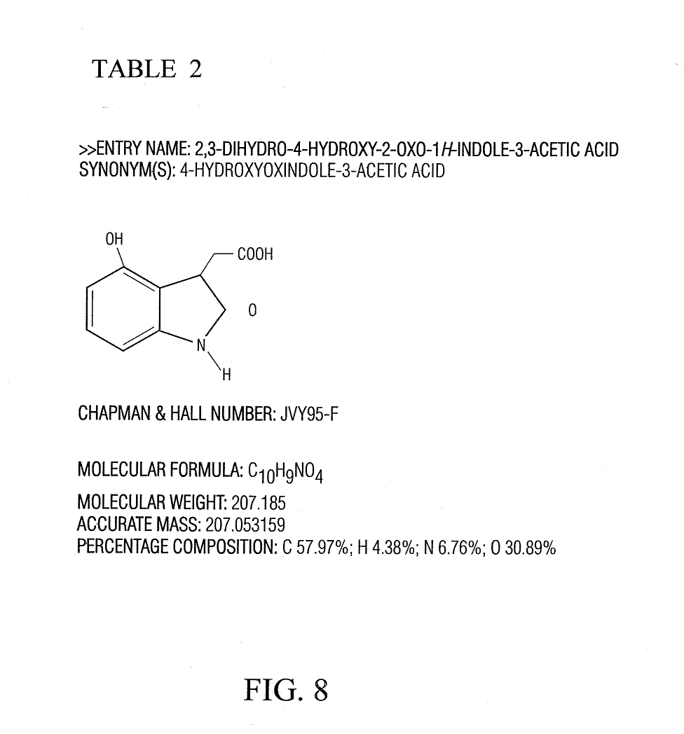

It was an objective of the present invention to demonstrate that purified Tasselin A, which is dervied from crude corn tassel alcoholic extracts, also inhibits melanin synthesis through inhibition of tyrosinase, a key enzyme in melanin synthesis. For this purpose, a modified assay method of anti-tyrosinase activity described in the literature was employed (Chang T S and Tseng M J., Mar. Sci. Technol., 14: 190-193, 2006). The assay employs mushroom tyrosinase enzyme to enzymatically convert a colorless solution tyrosine to a black melanin product. The anti-tyrosinase activity is measured by the reduction of black color development in the presence of the inhibitor, which in this instance is Tasselin A. FIG. 7 is a graph showing the inhibiting effect of two different concentrations of purified Tasselin A on tyrosinase activity as 50% reduction at a concentration of 150 microgram/ml over the 30 minute test end-point. These results show that Tasselin A (4-hydroxy-1-oxindole-3-acetic acid) would be a good candidate to serve as a skin whitening agent in a suitable cosmetic formulation.

### Example 7

### Skin Whitening Compositions

As expected from its extraction profile, Tasselin A is both water and methanol/ethanol soluble. Milligram quantities have been successfully lyophilized and stored as a dry powder under vacuum for up to a year. Given these physical properties, Tasselin A has been formulated in both lotion and cream as described in the Tables III, IV, and V below.

TABLE III
Tasselin A is formulated in an oil-in-water
(O/W) moisturizing lotion composition.

| INGREDIENT | Amount(% WT) |
|---|---|
| Part A | |
| Tasselin A | 0.2 |
| (4-hydroxy-1-oxindole-3-acetic acid) | |
| Glycerol | 5.0 |
| Benzalkonium Chloride | 0.13 |
| Distearyldimonium Chloride | 2.4 |
| Cornstarch (pure food grade) | 3.3 |
| Distilled Water | 79.4 |
| Part B | |
| Vegetable oils (EnviroGel PAC 100 | 9.0 |
| Citrus Grandis | 0.5 |
| (Grapefruit seed oil) | |
| Fragrance (optional) | 0.1 |

PROCEDURE: The method of formulating Tasselin A in an O/W emulsion system follows a standard two-step process. Table III above lists the is ingredients and their amounts for Part A and Part B. Part A is prepared by adding Tasselin A (0.1% to 5%, w/v) to the other water soluble components, which in this particular formulation are glycerol, benzalkonium chloride, distearyldimonium chloride, and natural corn starch powder. The starch slurry so formed is heated to 75-80 degree centigrade with continuous stirring to form a clear gel. Part B ingredients comprise a vegetable oil mix of hydrogenated Soy Bean oil, Cocoa Nucifera, and hydrogenated Cottonseed oil and Grapefruit seed oil, a natural preservative. Part B oils are then blended together at 75-80 degrees centigrade with the pre-heated gel until a homogeneous and semi-viscous creamy white emulsion is formed. The emulsion is cooled to room temperature.

TABLE IV
Tasselin A is formulated in an oil-in-water
(O/W) skin protecting barrier and moisturizing lotion
composition.

| INGREDIENTS: | Amount(% WT) |
|---|---|
| Part A | |
| Tasselin A | 0.2 |
| (4-hydroxy-1-oxindole-3-acetic acid) | |
| Glycerol | 5.0 |
| Benzalkonium Chloride | 0.13 |
| Distearyldimonium Chloride | 2.4 |
| Cornstarch (pure food grade) | 3.3 |
| Distilled Water | 75.4 |
| Part B | |
| Vegetable oils (EnviroGel PAC 100 | 9.0 |
| Dimethicone (DC 200 Fluid) | 0.8 |
| Dimethylcyclopentanesiloxane | 3.2 |
| (DC-245 Fluid) | |
| Citrus Grandis | 0.5 |
| (Grapefruit seed oil) | |
| Fragrance (optional) | 0.1 |

PROCEDURE: The method of formulating Tasselin A in an O/W emulsion system follows a standard two-step process. Table III above lists the is ingredients and their amounts for Part A and Part B. Part A is prepared by adding Tasselin A (0.1% to 5%, w/v) to the other water soluble components, which in this particular formulation are glycerol, benzalkonium chloride, distearyldimonium chloride, and natural corn starch powder. The starch slurry so formed is heated to 75-80 degree centigrade with continuous stirring to form a clear gel. Part B ingredients comprise a vegetable oil mix of hydrogenated Soy Bean oil, Cocoa Nucifera and hydrogenated Cottonseed oil, dimethicone, dimethylcyclopentane siloxane, and Grapefruit seed oil, a natural preservative. Part B oils are then blended together at 75-80° centigrade with the pre-heated gel until a homogeneous and semi-viscous creamy white emulsion is formed. The emulsion is cooled to room temperature with gentle stirring.

TABLE V
Tasselin A is formulated in an water-oil
(W/O) skin depigmenting cream.

| INGREDIENTS: | Amount(% WT) |
|---|---|
| Phase A | |
| Tasselin A | 0.2 |
| (4-hydroxy-1-oxindole-3-acetic acid) | |
| Glycerol | 6.0 |
| Guar gum | 0.3 |
| Distilled Water | 63.0 |
| Phase B | |
| Polyglyceryl-3-methylglycerol | 3.0 |
| Glyceryl Stearate | 2.0 |
| Stearyl alcohol | 1.0 |
| Caprylic/Caproic Triglyceride | 8.0 |
| Ethylhexyl Palmitate | 7.0 |
| Decyl Cocoate | 5.0 |
| Macaterndemia ternifolia nut oil | 3.0 |
| Tocopheryl acetate | 1.0* |
| Phase C | |
| Preservative, Citrus Grandis | 0.5 |
| (Grapefruit seed oil) | |
| Fragrance (optional) | q.s. |

PROCEDURE: Heat phase A and B separately to approximately 70-75° C. Add phase B to phase A with stirring. *Homogenize. Cool with gentle stirring.

*Important: charge the vessel with phase B first, phase A must be added without stirring.

TABLE VI
Tasselin A is formulated in as skin
depigmenting a deodorant stick.

| INGREDIENTS: | Amount(% WT) |
|---|---|
| Tasselin A (dissolved in anhydrous ethanol) | 0.2 |
| (4-hydroxy-1-oxindole-3-acetic acid) | |
| Probutyl 14 (Croda) | 28.5 |
| Cyclohexasiloxane (and) Cyclopentanesiloxane | 5.1 |
| (DC345 Fluid) | |
| C12-15 alkylbenzoate (Finsolv TN, Innosoec) | 5.3 |
| Hydrogenated Caster oil (Castro Wax NF, CasChem) | 4.8 |
| Poly C10-30 Alkyl acrylate | 5.0 |
| (Intellimer IPA-13-1, Air Products) | |
| Stearyl alcohol (Crodacol S-95, Croda) | 28.0 |
| Aluminum zirconium tetra chlorohydrex Gly, | |
| (18.48% active). | |
| -AAZG-7167 (80% active) (summit Res. Lab) | 23.1 |
| Isopropylparaben, isobutylparaben, and | 0.2 |
| butylparaben (LiquaPar oil, ISP.) | |

Procedure:

Combine all the ingredients in the above order in a vessel while stirring and begin to heat.

Continue to stir and heat until all the ingredients are completely dispersed and uniform, cool the formulation. At 70-72° C. pour batch into proper mold.

There has thus been shown and described a novel skin whitening agent derived from aromatic corn tassel extracts. Through detailed and extensive chemical analyses the active skin whitening agent was identified as 4-hydroxy-1-oxindole-3-acetic acid and designed here as purified Tasselin A was shown to inhibit melanogensis in a sporulating mold culture and to inhibit tyrosinase in an in vitro anti-tyrosinase enzyme assay. Tasselin A was also shown to have multi-functional biological and biochemical properties including potent antioxidant and anti-irritant activities. It was speculated that Tasselin A inhibits melanin synthesis through a competitive substrate-product inhibition reaction due to its structural similarity to the proximate precursor of melanin in the melanin biosynthesis pathway. On the basis of its demonstrated anti-tyrosinase activity four different compositions were presented and resides in the given composition and formulations of the preferred embodiments of the present invention. It is to be understood, that the examples of lotion, creams and deodorant sticks given in the above examples are not limited to those alone but can be any of the many oil-in water (O/W) and water-in-oil (W/O) formulations, and allied cosmetic applications available to one familiar with the state of the art in cosmetic compositions. Many changes, modifications, variations and other uses and applications of the subject invention will, however, become apparent to those skilled in the art after considering the specification and the accompanying changes, modifications, variations and other uses and applications which do not depart from the spirit and scope of the invention are deemed to be covered by the invention, which is not to be limited only by the claims which follow.

What is claimed is:

1. A cosmetic and dermatological composition comprising 2, 3-dihydro-2-oxo-1-H-indole-3-acetic acid as an active ingredient for use as an inhibitor of melanin production.

2. The composition defined in claim 1, wherein the active ingredient and related compounds are isolated and purified from corn tassel extracts.

3. The composition defined in claim 1, wherein the concentration range of the active ingredient is between 0.1 to 5.0 (% WT) of a preparation selected from the group consisting of a moisturizing skin lotion, a skin protecting barrier lotion, a skin cream, and a deodorant stick preparation.

4. The composition defined in claim 1, wherein the concentration of the active ingredient is between 0.2 and 2.0 (% WT) of a preparation selected from the group consisting of a moisturizing skin lotion, a skin protecting barrier lotion, a skin cream, and a deodorant stick preparation.

5. The composition defined in claim 1, wherein active ingredient serves as an anti-irritant.

6. The composition defined in claim 1, wherein the active ingredient serves as a skin whitening agent.

7. The composition defined in claim 1, wherein the active ingredient serves as a skin whitening agent.7A method of isolating and purifying Tasselin A compound (2, 3-dihydro-2-oxo-1-H-indole-3-acetic acid) comprising the steps of:
growing seed corn and harvesting the aromatic corn tassels' pollen sacs at the peak of aroma,
immersing the aromatic pollen sacs into a vessel containing ice-cold anhydrous ethanol,
forming an liquid alcoholic extract of the mixture by stirring for 24-48 hours at 4° C.,
clarifying the extract by low speed centrifugation and sterilizing the extract by filtering through a 0.4 micron filter into a sterile container,
concentrating the sterile extract a minimum of ten to twenty fold under vacuum by rotary evaporation at room temperature to obtain an final enrichment of 5-10% of the purified compound,
loading the concentrate onto a suitable chromatographic bed material and eluting the predominant phenoliuc peak, thereby separating the claimed compound from the bulk mass by the technique of preparative HPLC, and g) repeating steps (a) through (f) to collect an effective amount of the purified compound for use as an skin whitening ingredient in cosmetic and dermatological compositions.

8. The method defined in claim 7, wherein the elution is performed for approximately 15 minutes.
